(19) United States
(12) Patent Application Publication
(10) Pub. No.: US 2023/0158345 A1
(43) Pub. Date: May 25, 2023

(54) MULTI-MASK EQUIPMENT

(71) Applicant: MED INTERVENTIONS AND BEYOND PVT. LTD., Margao, Goa (IN)

(72) Inventor: Deepak PATHANIA, Margao (IN)

(73) Assignee: MED INTERVENTIONS AND BEYOND PVT. LTD., Margao, Goa (IN)

(21) Appl. No.: 17/919,350

(22) PCT Filed: Apr. 8, 2021
(86) PCT No.: PCT/IN2021/050350

§ 371 Date: Oct. 17, 2022

(30) Foreign Application Priority Data
Apr. 16, 2020 (IN) 202021016521

(51) Int. Cl. A62B 18/08; A62B 7/10; A41D 13/11; A62B 18/10

(52) U.S. Cl. CPC A62B 18/084; A62B 7/10; A41D 13/1184; A62B 18/10

(57) ABSTRACT

Described herein is a multi-mask equipment (100) that includes a mask assembly M configured to accommodate a filter assembly C, D and to mount a face protective shield H. The equipment enables a filter-based mask and shield can be used together as one integrated unit, and at the same time is future ready with more options to mount or use various other accessories, like oxygen inlet/outlet, electronic equipment etc. The multi-mask equipment 100 can be mounted over the head of a wearer by means of headbands as mounting mechanism F(LHS and RHS) and straps as adjusting mechanism A3. The equipment ensures that the overall cost of the mask and face protective shield assembly is substantially low as the equipment requires bare minimum recurring cost.

### CROSS-REFERENCE TO RELATED APPLICATIONS AND PRIORITY

The present application claims priority from Indian patent bearing application number 202021016521 filed on Apr. 16, 2020

### TECHNICAL FIELD

The present subject matter in general relates to a Personal Protective Equipment for Healthcare and public use, and in particular relates to a modular mask assembly that can facilitate modularity that can change the functionalities of the mask and facilitate adaptability of various kinds of filters and facilitate accessorization that include a face shield as well as various other accessories to perform a high range of functions, whenever required.

### BACKGROUND

Conventionally, various types of personal protective devices are known in the art, such as masks and face shields. Generally, masks are known to be categorized in 2 major categories, i.e., structured masks having a rigid body and a filter through which a person breathes, and masks having soft material that can be stretched across the face and tied behind ears or head of the user.

One of the main objectives of a mask is to ensure that the wearer does not transmit infection to others, particularly to patients who may already be suffering from ailments. Moreover, a mask protects the wearer from getting infected by inhaling air borne infectious particles, if any, from other persons and/or those suspended in air. Usually masks as well as filters mounted in the masks are designed according to the nature of particles being dealt with.

Face shields are known to be configured in many types, ranging from a transparent visor that covers face of the wearer and is attached to the head by a band, and a structure that rests on the forehead and is secured around the head with an elastic or rigid band. Other types of shields are also known in the art that function as a mask as well as seal the full face of the wearer, thereby enabling the wearer to breathe through filters in the shield.

The main purpose of a shield is to protect the wearer from any particles that might land on the wearers face by way of sneezing or coughing of a patient on the wearer and other such instances. Covid19 pandemic threw light on the fact that a virus may also enter the human system through ears apart from mouth, nose, and eyes. In other words, when a person with contaminated hands touches his/her face, eyes, nose or ears, the virus may enter the body through these parts and reach lungs.

For instances like Covid19 pandemic, due to the nature of the virus, both the mask and the shield are worn together, especially by healthcare professionals like doctors, and nurses, during inter-actions, diagnostics, patient care etc. Existing masks and shields work well, however, both are independent products and are used as independent products. The masks and shields were never designed to work like a single system of products.

Moreover, the existing masks and shields face a plurality of challenges, including but not limited to:

Usage: When the time comes to use masks and/or shields, a user has two independent products that have to fit on the head. It is disheartening to even imagine the plight of health workers who have to deal with a multitude of straps around their heads and keep adjusting one at the cost of the other one being disturbed. More particularly for women with long hair that is tied in a bun or ponytail, these multitude of straps can be even more painful.

Communication: Interaction of a user, especially a health worker, upon wearing a mask and a shield in front of his/her mouth is another cause of concern as the voice coming past two layers of obstacles over the mouth hardly reaches others and sometimes enormous efforts are required by the wearer to communicate with others. Therefore, a wearer has to stress a lot more every time they need to say something to patients. The lives of healthcare workers are understandably already stressed, and if each item of stress can be removed from their lives, it will be a great service to them.

Waste generation: With complete masks that are disposable, a lot of wastage is generated every day. Huge numbers of masks are used and thrown in hospitals, clinics, clinical laboratories etc. every single day and the bio-waste repercussions of such disposal are unimaginable.

Material efficiency: Less we consume today, the better it is. Instead of full masks which can be used and thrown, reusable masks which use replaceable filters are preferred. In such reusable masks, only the filters have to be thrown away and replaced after their usage cycle is over. This means an approximate increased efficiency of the same material by about 4-6 times. This also means less disposing of bio-waste by at least 4-6 times.

Currently if a healthcare worker, such as a doctor, uses a structured mask and not a soft material mask, then he/she cannot even wear a shield because the structured masks protrude out too much from the face. Further, existing mask and shield combination as separate products cannot be integrated with a fully sealed suit like the hazmat suit, which results in a need to procure a 3rd product. Moreover, upfront cost of the combination of existing mask and shield appear to be economical, however, even if these are to be used for a week, then the recurring costs increase exponentially.

Therefore, there is a well felt need for an apparatus and system that overcomes the above and other drawbacks relating to personal protective equipment.

### SUMMARY

It is an object of the present subject matter to reduce the efforts and inconvenience, especially health workers like doctors and nurses, in wearing face masks and face shields substantially.

It is another object of the present subject matter to enhance communication of health workers wearing face masks and face shields with co-workers and patients.

It is yet another object of the present subject matter to reduce the biomedical waste generation substantially.

It is yet another object of the present subject matter to improve the material efficiency in medical industry.

It is yet another object of the present subject matter to ensure that filter is not contaminated easily by foreign particles, including particles/droplets projecting from other person's cough and sneeze.

It is yet another object of the present subject matter to minimise chances of contamination of the modular apparatus by hand of the wearer.

It is yet another object of the present subject matter to provide a reusable and cost-effective equipment for protecting healthcare workers as well as patients from viruses and infections.

It is yet another object of the present subject matter to provide an apparatus that integrates a face mask, a face shield, and additional accessories.

It is yet another object of the present subject matter to provide a modular equipment in which a face shield can be attached on a face mask structure directly without additional straps.

It is yet another object of the present subject matter to provide a modular equipment in which face shield can simply be slipped on or removed without the requirement of bolting, strapping, locking or the like.

It is yet another object of the present subject matter to provide a modular equipment in which a face shield can be mounted on or removed from the face mask while on the move.

It is yet another object of the present subject matter to provide a modular equipment in which a face shield can be mounted on or removed from the face mask with ease by utilizing only one hand.

It is yet another object of the present subject matter to provide a modular equipment that can be worn by a user without straps going around the head or ears.

It is yet another object of the present subject matter to provide a modular equipment that is configured such that arms of spectacles can go past the headband of the apparatus without touching it during use.

It is yet another object of the present subject matter to provide a modular equipment that can be worn even with the biggest standard spectacles.

It is yet another object of the present subject matter to provide a modular equipment that is configured to cater to spread of new pandemics/diseases.

It is yet another object of the present subject matter to provide a modular equipment that is configured to employ electronic filters merely by providing a small additional fitment.

It is yet another object of the present subject matter to provide a modular equipment in which a cartridge-based filtration system can be employed as a modular fitment.

It is yet another object of the present subject matter to provide a modular equipment in which sheet-based filter can be employed to provide a cheap, quick, and easily accessible filter assembly.

It is yet another object of the present subject matter to provide a modular equipment in which the face mask can be made of transparent, translucent, or opaque materials.

It is yet another object of the present subject matter to provide a modular equipment that allows modularity in visual appeal.

It is yet another object of the present subject matter to provide a modular equipment that is configured to accommodate face shields of different sizes and thickness.

The subject matter relates to a multi-mask equipment comprising a mask assembly configured to be mounted on at least mouth and nose of a wearer and comprising at least one mount, to removably attach different modules that could change the entire functionality of the mask itself, a removable face protective shield or other accessories, a filter assembly which can accommodate filters of various kinds, and at least one mounting mechanism for mounting the Multi-Mask equipment on the head of a wearer.

In an embodiment, the mask assembly comprises a hard component for housing modules that can be attached for changing the functionalities like oxygen inlet/outlet to be used with ventilators etc., or an electronic equipment on the face, or an electronic filter, or a fitment for voice enhancement module, or an oxygen mask attachment etc., the open ended structure being such that it allows endless possibilities of adding new modules.

In another embodiment of the present subject matter, the mask assembly comprises a hard component for housing components of the at least one filter assembly that can be adapted to easily facilitate any filter materials or systems including cloth, n95, HEPA or even a cartridge based system etc., the open ended structure being such that it allows endless possibilities of adding new modules.

In another embodiment of the present subject matter, a shield or another protective accessory is configured to be simply slipped into the groove of the mask as and when required without any additional clipping or bolting, and removed as simply as pulling it upwards and off the hard component that covers a part of the face as required.

In another embodiment of the present subject matter, the mask assembly comprises a hard component providing the structure to facilitate modularity, accessorisation and filter adaptability, transforms or is attached to a soft pliable component that conforms to contours of the face of the wearer to make an airtight seal when the Multi-Mask equipment is worn by the wearer.

In yet another embodiment of the present subject matter, the mask assembly is configured as a cup shaped assembly comprising at least one wall and defining a front mask opening in the hard component.

In yet another embodiment of the present subject matter, at least one cavity is provided on the at least one wall for accommodating an exhalation valve or any other exhalation accessory.

In yet another embodiment of the present subject matter, a closure component is provided to be removably attached and locked in place with the at least one wall comprising one or more raised parts for covering the front mask opening.

In yet another embodiment of the present subject matter, the closure component comprises one or more vents to enable the wearer to breathe in air.

In yet another embodiment of the present subject matter, the closure component comprises one or more protrusions for enabling the wearer to hold the mask assembly during attachment or detachment of the face protective shield and/or other components, attachments, or accessories on the mask assembly.

In yet another embodiment of the present subject matter, the at least one filter assembly comprises a filter grille and a filter material and is housed inside the mask assembly.

In yet another embodiment of the present subject matter, the mask assembly comprises at least one slit on its circumference for mounting the at least one face protective shield thereon.

In yet another embodiment of the present subject matter, the at least one face protective shield comprises at least one shield opening for mounting the face protective shield on the mask assembly.

In yet another embodiment of the present subject matter, the at least one mounting mechanism comprises at least one left-side headband and at least one right-side headband attached to each other by at least one locking mechanism.

In yet another embodiment of the present subject matter, at least one of the at least one left-side headband and at least one right-side headband comprises at least one set of teeth for adjusting the left-side headband and the right-side headband with respect to each other, thereby enabling mounting of the multi-mask equipment on heads of various sizes and shapes.

In yet another embodiment of the present subject matter, at least one of the at least one left-side headband and at least one right-side headband comprises at least one set of connectors.

In yet another embodiment of the present subject matter, the multi-mask equipment further comprises at least one adjusting mechanism for adjusting the position of the mask assembly on the face of the wearer.

In yet another embodiment of the present subject matter, the at least one adjusting mechanism comprises a plurality of belts, each belt of the plurality of belts being secured to the structured mask assembly at one end and configured to engage with the connectors on the at least one headband at the other end.

In yet another embodiment of the present subject matter, the at least one adjusting mechanism comprises a plurality of teeth for engaging with connectors on the at least one left-side headband and at least one right-side headband.

### BRIEF DESCRIPTION OF ACCOMPANYING DRAWINGS

The present invention, both as to its organisation and manner of operation, together with further objects and advantages, may best be understood by reference to the following description, taken in connection with the accompanying drawings. These and other details of the present invention will be described in connection with the accompanying drawings, which are furnished only by way of illustration and not in limitation of the invention, and in which drawings:

FIG. 1 illustrates a three-dimensional view of a Multi-Mask equipment in accordance with one embodiment of the present subject matter.

FIG. 2 illustrates an exploded view of the Multi-Mask equipment in accordance with one embodiment of the present subject matter.

FIG. 3 illustrates a front view and a side view of a Multi-Mask equipment in accordance with one embodiment of the present subject matter.

FIG. 4 illustrates side views of the Multi-Mask equipment with additional attachments according to different embodiments of the presents subject matter.

FIG. 5 illustrates a side view of the multi-mask equipment with a magnified representation of groove between the wall and the hard surface in accordance with one embodiment of the present subject matter.

### DETAILED DESCRIPTION

The following presents a detailed description of various embodiments of the present subject matter with reference to the accompanying drawings.

The embodiments of the present subject matter are described in detail with reference to the accompanying drawings. However, the present subject matter is not limited to these embodiments which are only provided to explain more clearly the present subject matter to a person skilled in the art of the present disclosure. In the accompanying drawings, like reference numerals are used to indicate like components.

The specification may refer to “an”, “one”, “different” or “some” embodiment(s) in several locations. This does not necessarily imply that each such reference is to the same embodiment(s), or that the feature only applies to a single embodiment. Single features of different embodiments may also be combined to provide other embodiments.

As used herein, the singular forms “a”, “an” and “the” are intended to include the plural forms as well, unless expressly stated otherwise. It will be further understood that the terms “includes”, “comprises”, “including” and/or “comprising” when used in this specification, specify the presence of stated features, integers, steps, operations, elements, and/or components, but do not preclude the presence or addition of one or more other features, integers, steps, operations, elements, components, and/or groups thereof. It will be understood that when an element is referred to as being “attached” or “connected” or “coupled” or “mounted” to another element, it can be directly attached or connected or coupled to the other element or intervening elements may be present. As used herein, the term “and/or” includes any and all combinations and arrangements of one or more of the associated listed items.

The figures depict a simplified structure only showing some elements and functional entities, all being logical units whose implementation may differ from what is shown.

The present subject matter relates to a personal protective equipment that solves the problems of conventional masks and shields.

Usage: The personal protective equipment according to the present subject matter comprises a modular structure in which a face mask and a face shield are integrated. The present equipment also provides a provision to integrate various other accessories, such as accessories for oxygen inlet/outlet, electronic equipment etc. with the face mask. In the present invention, the facemask is configured such that it becomes a chassis to load on additional accessories, particularly the face shield. More about this modularity is explained in the following paragraphs.

Communication: Since the face shield in the present personal protective equipment is configured to be fitted to the structure of the face mask, it is preferably provided with a big hole/opening which is covered by the filter of the face mask upon assembly. So now in the structure according to the present subject matter, the voice of the wearer needs to come through only one set of porous material, and that is the filter. Hence, the voice of the wearer is exponentially more audible as compared to existing face masks and/or their combination with face shields.

Waste generation: All the parts of the present personal protective equipment can be made of regular plastic moulded material that can be used for a long time repeatedly. The only waste that is required to be disposed of after their cycle of use are the filters.

Material efficiency: Conventionally, it is very difficult to wear structured masks with face shields and therefore, the face mask had to be soft type. This led to disposal and wastage of a lot more material than is actually needed to be used. The vital reusable part of the present personal protective equipment is the filter, which is a usage component of only ¼th or ⅙th the soft type material mask.

According to a preferred embodiment of the present invention, the filter-based mask and shield can be used together as one integrated unit. Integration with hazmat or any other suits can be done by slight modification of the face shield which allows fitment to the suit. Therefore, the equipment according to the present subject matter is not just future ready but open ended for a lot more options.

Moreover, the overall cost of the present equipment is substantially low as the equipment requires bare minimum recurring cost.

One of the biggest advantages of the present subject matter is the modularity and therefore, the “open-endedness” of the equipment that makes it future ready for future attachments and accessories. This can be understood by the fact that the equipment according to the present subject matter can be configured as but not limited to the following:
1. A structured mask with a filter.
2. A structured mask without a filter.
3. A structured mask with a shield.
4. A structured mask with inlet and outlet of gases, such as oxygen.
5. A structured mask system that can be fitted to a completely sealed suit, or a hazmat suit.
6. A structured mask with a changeable type of filters to accommodate different filtration systems like the ones to protect from toxic gases etc.
7. A structured mask with an electronic system inbuilt to accommodate camera and other video and sound recording devices

The personal protective equipment according to the present subject matter is configured as a multi-mask or a structured mask in a preferred embodiment. Therefore, the terms ‘personal protective equipment’ and ‘multi-mask equipment’ are interchangeably used hereinafter in the present description. The equipment according to the present subject matter is highly beneficial to be used primarily by all healthcare professionals including and not limited to, doctors, nurses, admin, housekeeping and even patients. However, the use of the personal protective equipment according to the present subject matter can further be extended to various applications because of its inherent capacity of being modular. Some of the applications of the present personal protective equipment include but not limited to the following:
1. Policemen, especially during pandemics or in high pollution areas.
2. Healthcare workers and first responders who go to affected areas.
3. Fire brigade officials.
4. Professionals involved in services which require interaction with strangers and yet need to be protected during epidemics or pandemics, like airline/train staff, shopkeepers, delivery persons, travelers, etc.
5. Painters and people who work with fumes and pollution.

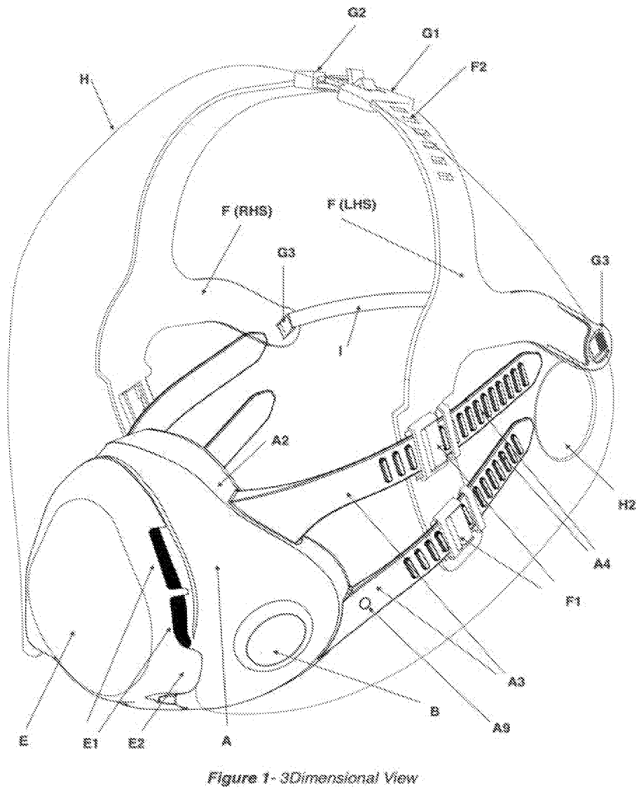

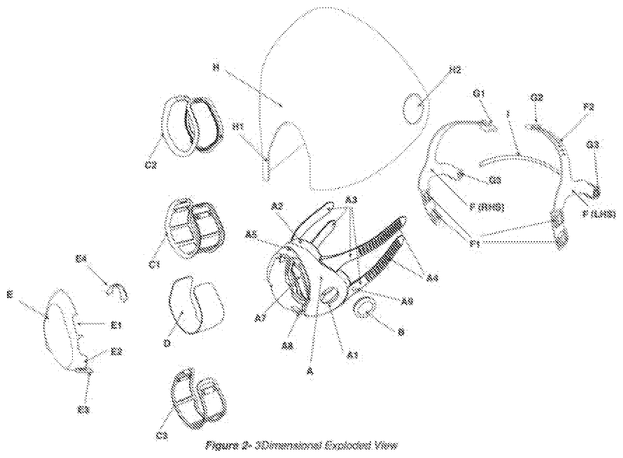

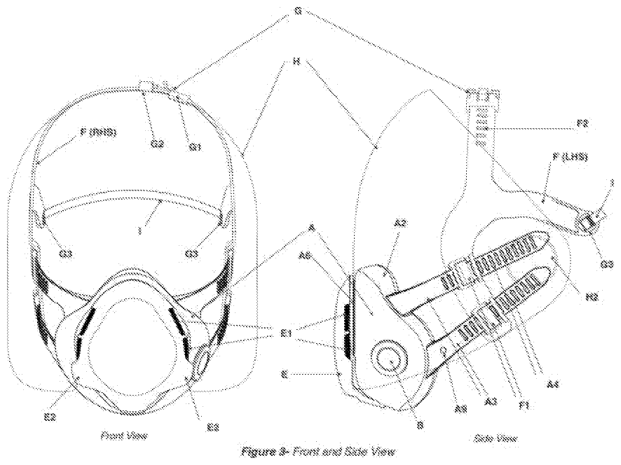

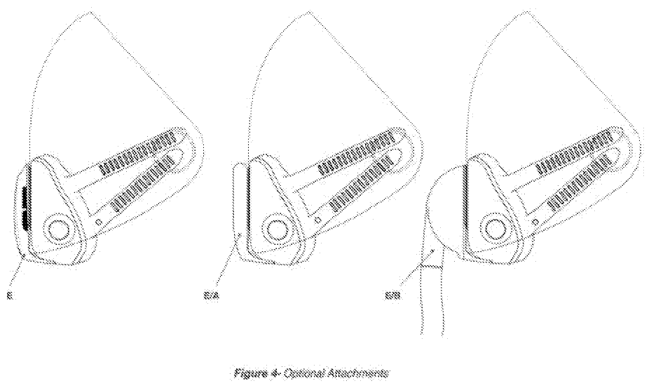

FIGS. 1 and 2 illustrate a three-dimensional view and an exploded view of a Multi-Mask equipment 100 in accordance with one embodiment of the present subject matter. FIG. 3 illustrates a front view and a side view of the Multi-Mask equipment 100 in accordance with one embodiment of the present subject matter. FIG. 4 illustrates side views of the Multi-Mask equipment 100 with additional attachments according to different embodiments of the presents subject matter. The Multi-Mask equipment 100 may also be referred as Multi-Mask assembly 100 with-out departing from the scope of the present invention.

The Multi-Mask equipment 100 according to the present subject matter includes a plurality of components. For example and without limiting the scope of the present subject matter, major structural components of the Multi-Mask equipment 100 include a structured mask assembly M comprising a first component A, a cap B, and a closure component E, configured to be mounted on the mouth of a wearer and held by a mounting mechanism F that is configured to mount the Multi-Mask equipment 100 on the head of the wearer while locking it with a locking mechanism G, at least one adjusting mechanism A3 for adjusting the position of the structured mask assembly M on the face of the wearer, at least one filter assembly C, D positioned in the structured mask assembly M, and at least one face protective shield H. According to the present invention, the face protective shield H may or may not be used at all times as is also described hereinbelow. The structured mask assembly M forms the main “chassis” of and is the backbone to the Multi-Mask equipment 100. In a preferred embodiment, the structured mask assembly M comprises a first component A that is configured as a hard component of the structured mask assembly M and a second component A2 that is configured as a soft pliable component of the structured mask assembly M. The hard component A provides rigidity to the structured mask assembly M and houses various components of the filter assembly C, D. The soft pliable second component A2, on the other hand, adjusts to conform to the contours of the face of a wearer to make an airtight seal on the face when the Multi-Mask equipment 100 is worn by the wearer. In an embodiment, the soft pliable component A2 comprises face adjustability feature, i.e., the soft pliable component A2 is configured to fit people having different face profiles so that the soft pliable component A2 is snug fit thereupon. The soft pliable material A2 makes contact with the face of a wearer along its entire surface when the structured mask assembly M is worn by the wearer in an embodiment. In another embodiment, the hard component A and the soft pliable component A2 are integrated into one unit to form the base of the Multi-Mask equipment 100. However, in another embodiment, the hard component A and the soft pliable component A2 may be separate components such that they are capable of being replaced in case of any damage or wear and tear.

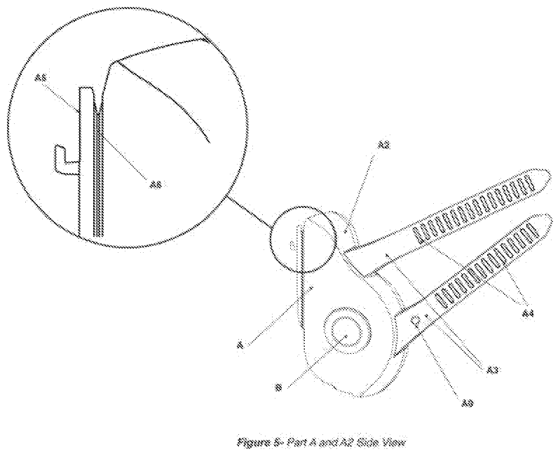

In an embodiment, the structured mask assembly M is configured as a cup shaped assembly, which covers the mouth and nose of the wearer. The cup shaped assembly of the structured mask assembly M comprises a front mask opening A7. At least one wall A5 is provided parallel to the front surface of the hard component A such that a mount comprising a groove A6 is defined between the front surface of the hard component A and the wall A5, as shown in FIGS. 1, 2, 3 and 5. FIG. 5 illustrates a side view of the multi-mask equipment with a magnified representation of groove 6 between the wall A5 and the hard surface A. The groove A6 is configured to mount the face protective shield H on the hard component A of the structured mask assembly M. In an embodiment, the front wall A5 partially covers the front surface of the hard component A to define the groove A6 there between. In another embodiment, the front wall A5 covers upper half of the front surface of the hard component A to define the groove A6 there between. In yet another embodiment, the front wall A5 covers the entire front surface of the hard component A to define the groove A6 there between. In yet another embodiment, more than one walls may be provided to define a groove between said walls and the front surface of the hard component A. For the purpose of present description, the wall A5 is also referred to as the front wall A5.

The hard component A and the soft pliable component A2 are aligned with each other in a back to back configuration. In an embodiment, the structured mask assembly M can be configured as a structured/moulded mask in the shape as shown in FIGS. 1 and 2. The hard component A of the structured mask assembly M may be configured in the form of a cylinder or a truncated cylinder or even have a curved or rectangular shape in different embodiments without departing from the scope of the present subject matter. The shape as well as physical design elements of the present structured mask assembly M can be altered regularly depending upon the requirements in different embodiments. For example, the shape and configuration of the structured mask assembly M can be varied keeping in mind new materials that are coming into use at a later date.

In an embodiment, the hard component A of the structured mask assembly M comprises at least one cavity A1 on the hard component A for accommodating an exhalation valve or any other exhalation accessory, if required as shown in FIG. 2. In an embodiment, a cavity A1 is provided on one side at the circumference of the hard component A. In another embodiment, more than one cavity A1 may be provided along the circumference of the hard component A. In a preferred embodiment, a cap or plug B comprising a soft pliable material is provided to close the cavity A1 formed in the hard component A when the cavity A1 is not required.

In a preferred embodiment, the front section of the structured mask assembly M is open such that the wall A5 define the front mask opening A7. The front mask opening A7 in the front section of the structured mask assembly M comprises a fitment centre. In an embodiment, the structured mask assembly M comprises one or more provisions to fix a closure component E and/or additional attachments or accessories with the hard component A.

In an embodiment, the closure component E is configured to be removably attached and/or locked in place with the wall A5 to cover the front mask opening A7. In this regard, one or more attachment means and/or locking means are provided in the wall A5 as well as on the closure component E. In an embodiment, the closure component E comprises one or more protrusions E3 at the bottom that engages with a counter fitment A8 provided on the inner side of the hard component A. In a preferred embodiment the counter fitment A8 is provided at the bottom of the hard component A. In an embodiment, the closure component E comprises one or more vents E1 on either side to enable the wearer to breathe the air in. The closure component E also comprises one or more protrusions E2 on either side for enabling the user to hold on to the structured mask assembly M while attaching or detaching at least one face protective shield H and/or other components, attachments, or accessories.

The face protective shield H according to the present embodiment is configured as a face visor. However, in other embodiments, the face protective shield may comprise one or more goggles or any other transparent, partially transparent or translucent component or an assembly of components that are configured to protect the face or a part of the face of the wearer from foreign materials comprising but not limited to flying objects and road debris, chemical splashes, or potentially infectious materials. In a preferred embodiment, as shown in FIG. 2, the face protective shield H comprises at least one shield opening H1 for enabling mounting of the face protective shield H on the structured mask assembly M.

In a preferred embodiment, the groove A8 defined between the front surface of the hard component A and the front wall A5 is configured for mounting the face protective shield H on the structured mask assembly M. The groove A6 according to the present embodiment is configured to align with the shield opening H1 of the face protective shield H such that the face protective shield H is secured on the structured mask assembly M during assembly, as shown in FIG. 1. The shield opening H1 and the groove A6 of the present embodiment are configured such that the face protective shield H can be slid from the top of the structured mask assembly M to mount said face protective shield H on the structured mask assembly M. However, in another embodiment, the shield opening H1 and the groove A6 may be configured such that the face protective shield H can be mounted on the structured mask assembly M from the front, bottom and/or either side of the structured mask assembly M.

In an embodiment, the face protective shield H comprises one or more depressions H2 on one or either side for securing a label or personalisation of the Multi-Mask equipment 100 or branding. The present invention enables a user to easily attach or detach the face protective shield H from the structured mask assembly M even when the structured mask assembly M is worn by the user. This is done by merely holding the structured mask assembly M and in particular, by holding the protrusions E2 provided on the closure component E by one hand and attaching or detaching the face protective shield H from the other hand in an embodiment.

The structured mask assembly M according to the present subject matter is configured to accommodate at least one filter assembly C comprising but not limited to a filter grille sub-assembly C1, C2, C3 and a filter material D. In a preferred embodiment, the front mask opening A7 houses the filter assembly C inside it. This deep setting of the filter assembly ensures that the filter assembly is secured inside the structured mask assembly M and inadvertent touching of the filter assembly by a wearer with contaminated hands is prevented. In a preferred embodiment, the filter assembly C is configured to occupy the space in the front mask opening A7 and forms the support for the filter material D. In the present embodiment, the filter assembly C has a curved configuration comprising a first frame member C1 that is covered by a soft pliable second frame member C2 and a third frame member C3. The filter grille sub-assembly C1, C2, C3 is configured to accommodate the filter material D therein. The filter material D of the present embodiment also comprises a curved configuration to match with the configuration of the base frame member or inner grille C1 such that the filter material D is securely mounted between the first frame member or grille C1 and the soft pliable second frame member or gasket C2 and held securely by the third frame member or outer grille C3. In another embodiment, the configuration of the filter grille sub-assembly C1, C2, C3 and the filter material D may vary to accommodate the same inside the hard component A and provide most effective filtration feature. In an embodiment, one or more provisions are provided on the inner side of the hard component A to secure the filter sub-assembly C of the filter assembly. The filter material D could be of any material that is required and relevant to the need since the mask could be used for any purpose.

In an embodiment, the structured mask assembly M is configured to be mounted on the head of a user by means of at least one set of mounting mechanism F. In a preferred embodiment, the mounting mechanism comprises at least one pair of headbands F (LHS and RHS) attached to the adjusting mechanism A3, which is further attached to the soft pliable material A2 of the structured mask assembly M. In an embodiment, the pair of headbands comprises at least one left-side headband F(LHS) and at least one right-side headband F(RHS) attached to each other by at least one locking mechanism G1 and G2 at two places. In an embodiment, a first locking mechanism G1 is provided on the right-side headband F(RHS) and a second locking mechanism G2 is provided on the left-side headband F(LHS). In an embodiment, the locking mechanisms G1, G2 comprise a first connector G1 and a second connector G1. In a preferred embodiment, the right-side headband F (RHS) comprises a C-shaped configuration with a set of first connectors G1 provided at the ends of the two long projections and the left-side headband F (LHS) comprises a set of reverse C-shaped configuration with a set of second connectors G2 to hold and slide the right-side headband F (RHS). A set of teeth/projection are provided on the first connectors G1 on the right-side headband F (RHS) to engage with a plurality of slots F2 on the left-side headband F (LHS) to lock the two sides of the headband on the head. The configuration of the projection is such that the headbands F (LHS and RHS) can be pushed into each other freely with the lock slipping past the slots F2 as it tightens on the head but the moment it is pulled apart, the mechanism locks itself in a preferred embodiment. To loosen the headbands F (LHS and RHS), the projection would need to be pulled up slightly so that it disengages with the slot and then it is free to move apart, thereby enabling mounting of the multi-mask equipment 100 on heads of various sizes and shapes.

In an embodiment, the mounting mechanism comprising the headbands F (LHS and RHS) are further configured to be adjusted on the head of a wearer by means of the adjusting mechanism A3. In a preferred embodiment, the adjusting mechanism comprises a plurality of belts A3, each belt A3 being secured to the structured mask assembly M at one end and configured to engage with the connectors F1 provided on the bottom section of the headbands F (LHS and RHS) on the part that comes on the face. A plurality of teeth/projections A4 is provided on each belt A3 that engage with the connectors F1 to adjust the position of the headbands F with respect to the structured mask assembly M on the head of a wearer. As shown in FIGS. 1, 2 and 3, the belts A3 are provided on either side of the structured mask assembly M in an embodiment. The belts A3 go on to the side of face of the wearer and connect to the headbands F (LHS and RHS) respectively at connecters F1 with clips that hold the belts A3 in such a way that when it is pulled through the clips then it is free to go through but the moment the belt A3 tries to loosen itself, the clip engages the lock and the belt A3 gets locked in place. This way the mask assembly M is tightened over the face of the wearer.

The headbands F (LHS and RHS) on either side are attached through the locking mechanism G1, G2 at two places, as shown in FIG. 1. This configuration allows the headbands F (LHS and RHS) to fit on heads of various sizes and shapes. In the present embodiment, the mounting mechanism F and the adjusting mechanism A3 are configured as separate components. However, in another embodiment, the adjusting mechanism A3 may be configured as an integral part of the mounting mechanism F. In yet another embodiment, the mounting mechanism F itself may act as an adjusting mechanism to adjust the position of the structured mask assembly M on the head of a wearer. In yet another embodiment, the headband F(LHS and RHS) could also be done away with and the straps A3 could be held around the head using extensions that stretch around the head and hold the straps A3 using the teeth/projections A4 for grip, in the same way as it locked in the clip at F1. In an embodiment, a hole A9 is provided in each of the lower two straps A3 so that incase the wearer wants to tighten the mask on the face even more, these holes A9 can be used to pass an elastic through them which goes around the neck of the wearer and is then secured behind the neck.

In an embodiment, the Multi-Mask equipment 100 comprises a rectangular clip G3 with fine teeth within the circular framework as shown in FIGS. 1, 2 and 3. The clip G3 is configured such that it is attached to the circular framework on one side of the rectangle and free on the other three sides. The teeth of the clip G3 are provided on the opposite side to where it is attached. This means that the side with the teeth can rise above the surface, pivoted at the point of attachment. The clip G3 is configured to rise only in one direction, i.e. in upward direction in an embodiment. The clip G3 cannot go downwards because then the teeth will hit the front wall. The gap between the teeth and the wall of the frame in front of it is very tiny and an elastic strap I is pushed in from below. Once the free end of the strap I comes through above the clip G3 and its frame, then a user can easily pull the free end to tighten the strap I. However, the moment strap I is pulled from the bottom end that goes around the head, the teeth of the clip G3 digs in and engage with the strap I, pushing the clip G3 against the front wall and hence, locking it in position. In a preferred embodiment, this mechanism is provided on both sides of the head, i.e. on left and right side of the headband. This way it becomes easy for the user to tighten the elastic straps on both sides of his head, and yet secure it in the position that the user stops tightening.

The Multi-Mask equipment 100 according to the present subject matter enables a wearer to use any other accessory while the Multi-Mask equipment 100 is in use. In an embodiment, if oxygen needs to be fed to a user wearing the Multi-Mask equipment 100, the closure component E and the grille C along with the filter material D can be removed. Once these are removed, a new oxygen fitment E/B can be attached on the same mount where the filter closure component E was located, as shown in FIG. 4. Similarly, in another embodiment, if a voice enhancing electronic module needs to be installed instead of the oxygen feeder, the oxygen feeder can be removed and can be replaced with the required module E/A as shown in FIG. 4. There would be an endless number of fitments that could be designed anytime to be fitted onto the Multi-Mask equipment 100. Further, it is also possible to mount any additional fitment with or without the face protective shield H in different embodiments.

Furthermore, various designs of face protective shields could be made and employed in the Multi-Mask equipment 100 of the present invention, depending on the requirement. The examples of face protective shields which may be fitted with the Multi-Mask equipment 100 include but not limited to shields covering full face, only eyes or any other part on the face, transparent, translucent, opaque shields. Moreover, various optional attachments are depicted in FIG. 4, wherein ‘E/A’ comprises a fitment for voice enhancement module other such requirement and ‘E/B’ is an oxygen mask attachment.

The Multi-Mask equipment 100 according to the present subject matter provides a plurality of advantages, including but not limited to the following:
1. The face protective shield can be attached on the mask structure directly without any additional straps.
2. The face protective shield can simply be slipped on or removed without any additional requirement bolting, strapping, locking etc.
3. The face protective shield can be added on or removed while on the move.
4. The face protective shield can be added on or removed with one hand.
5. The headband secures the mask without straps going around the head or ears with or without face protective shield.
6. The headband secures the mask in a way that a user needs to adjust the mask on his face only once and after that it is easy to wear or remove the mask while retaining the same adjustments.
7. The headband secures the mask in such a way that the mask can be worn or removed with one hand with or without face protective shield.
8. The headband is configured in such a way that the spectacle arms can go past the headband without touching it, thereby making it easy to wear or remove the spectacles while the mask is on.
9. The face protective shield can be worn even with the biggest standard spectacles.
10. The mask is configured in such a way that it is virtually impossible for the breath of the user to fog up the shield, while this is a common issue in most shields.
11. The structure enables modular fitment of various components of the Multi-Mask equipment, thereby making it easy to replace any single part that might be damaged.
12. The structure enables modular fitment of various functions of the Multi-Mask and this makes the mask structure deal with any new issues.
13. In case of a new pandemic where only certain electronic filters can be used, the Multi-Mask equipment only needs a small additional fitment with the electronic filter or any other accessory and its ready to be used.
14. For any new cause, the speed and cost to bring a new product to market will be shorter because in all conventional masks, the bigger problem to solve is not just the filter, but the fitment/ergonomic/securing of said masks on the face. The Multi-Mask equipment already addresses this.
15. Unlike many conventional masks which are moulded and utilise cartridge based filtration system, the Multi-Mask equipment according to the present invention can perform either, if required a cartridge system can be added as a modular fitment, but otherwise, using sheet based filter means the change of filter is cheap, quick and easily accessible.
16. The fitment of securing the straps to the headband is also especially configured for our need.
17. The mask is transparent and can easily be made in translucent or opaque materials.
18. Our mask allows modularity in visual appeal as well since the filter cap, the front part can be changed by users to suit their colour or aesthetic requirements.
19. The positioning of the filter is such that the droplets coming out of the mouth while talking, coughing or sneezing do not directly fall on the filter.
20. The filter is protected in front by a cap, and this makes sure no external projectile particles of the other person's cough or sneeze can land on the filter. This is important because what makes N95 type filters, 95% proof and not 100% proof are situations like this where a droplet falls on the filter and increases the chances of infection. In the present multi-mask, this chance is totally removed because for foreign particle to reach the filter, said particle has to take many twists and turns.
21. The shield fitment on the mask is done in such a way that if a droplet lands on the shield and trickles down, it cannot reach inside to the filter. On the contrary, the droplets will drop down to the ground.
22. There is a hole provided for the exhalation valve as well so that if the valve is required then that could also be fit easily. In the standard mask the hole is covered with a removable cap.
23. The shield receptacle on the mask is configured in such a way that it can take different thicknesses and sizes of shields without any change.
24. The circular dip on the sides of the shield, near the ears, can be configured for sticking a name tag or a branding sticker so that it does not come out easily.
25. The Multi-Mask equipment 100 comprises two small projections that serve as points to hold the mask while putting shield on or to adjust the mask. This is an important aspect so that the person does not keep touching the whole mask or different parts of the mask to help minimise any chance of contamination by hand.
26. The multi-mask materials are chosen for a wide range of sanitisation methods that involve dipping in liquids or UV sanitisation etc.
27. The filter assembly can be removed easily, and the filter changed or washed easily.

While the preferred embodiments of the present invention have been described hereinabove, it should be understood that various changes, adaptations, and modifications may be made therein without departing from the spirit of the invention and the scope of the appended claims. It will be obvious to a person skilled in the art that the present invention may be embodied in other specific forms without departing from its spirit or essential characteristics. The described embodiments are to be considered in all respects only as illustrative and not restrictive.

I/we claim:

1. A multi-mask equipment 100 comprising:
a mask assembly M configured to be mounted on at least mouth and nose of a wearer and comprising at least one mount, to removably attach different modules that could change the entire functionality of the mask itself, a removable face protective shield H or other accessories, a filter assembly which can accommodate filters of various kinds, and at least one mounting mechanism F(LHS and RHS) for mounting the Multi-Mask equipment 100 on the head of a wearer.

2. The multi-mask equipment 100 as claimed in claim 1, wherein the mask assembly M comprises a hard component A for housing accessories modules that can be attached for changing the functionalities like oxygen inlet/outlet to be used with ventilators etc., or an electronic equipment on the face, or an electronic filter, or a fitment for voice enhancement module, or an oxygen mask attachment etc., the open ended structure being such that it allows endless possibilities of adding new modules.

3. The multi-mask equipment 100 as claimed in claim 1 or 2, wherein the mask assembly M comprises a hard component A for housing components of the at least one filter assembly that can be adapted to easily facilitate any filter materials or systems including cloth, n95, HEPA or even a cartridge based system etc., the open ended structure being such that it allows endless possibilities of adding new modules.

4. The multi-mask equipment 100 as claimed in claim 1, wherein a shield or another protective accessory is configured to be simply slipped into the groove A6 of the mask as and when required without any additional clipping or bolting, and removed as simply as pulling it upwards and off the hard component A that covers a part of the face as required.

5. The multi-mask equipment 100 as claimed in claims 1, 2, 3 & 4, wherein the mask assembly M comprises a hard component A providing the structure to facilitate modularity, accessorisation and filter adaptability, transforms or is attached to a soft pliable component A2 that conforms to contours of the face of the wearer to make an airtight seal when the Multi-Mask equipment 100 is worn by the wearer.

6. The multi-mask equipment 100 as claimed in any one of claims 1 to 5, wherein the mask assembly M is configured as a cup shaped assembly comprising at least one wall A5 and defining a front mask opening A7 in the hard component A.

7. The multi-mask equipment 100 as claimed in claim 6, wherein at least one cavity A1 is provided on the circumference of the hard component A for accommodating an exhalation valve or any other exhalation accessory.

8. The multi-mask equipment 100 as claimed in any one of claims 6 and 7, wherein a closure component E is provided to be removably attached and locked in place with the at least one wall A5 for covering the front mask opening A7.

9. The multi-mask equipment 100 as claimed in claim 8, wherein the closure component E comprises one or more vents E1 to enable the wearer to breathe in air.

10. The multi-mask equipment 100 as claimed in any one of claims 8 and 9, wherein the closure component E comprises one or more protrusions E2 for enabling the wearer to hold the mask assembly M during attachment or detachment of the face protective shield H and/or other components, attachments, or accessories on the mask assembly M.

11. The multi-mask equipment 100 as claimed in any one of previous claims, wherein the at least one filter assembly comprises a filter grille sub-assembly C1, C2, C3 and a filter material D and is housed inside the mask assembly M.

12. The multi-mask equipment 100 as claimed in any one of previous claims, wherein the mask assembly M comprises at least one groove A6 between the wall A5 and the front surface of the hard component A along the circumference of the hard component for mounting the at least one face protective shield H thereon.

13. The multi-mask equipment 100 as claimed in any one of previous claims, wherein the at least one face protective shield H comprises at least one shield opening H1 for mounting the face protective shield H on the mask assembly M.

14. The multi-mask equipment 100 as claimed in any one of previous claims, wherein the at least one mounting mechanism F comprises at least one left-side headband F(LHS) and at least one right-side headband F(RHS) attached to each other by at least one set of sliding and locking mechanism G1 and a slider mechanism G2.

15. The multi-mask equipment 100 as claimed in claim 14, wherein at least one of the at least one left-side headband F(LHS) and at least one right-side headband F(RHS) comprises at least one set of slots F2 for adjusting the left-side headband F and the right-side headband F with respect to each other, using the locking mechanism G1, thereby enabling mounting of the multi-mask equipment 100 on heads of various sizes and shapes.

16. The multi-mask equipment 100 as claimed in any one of claims 14 and 15, wherein at least one of the at least one left-side headband F(LHS) and at least one right-side headband F(RHS) comprises at least one set of connectors F1.

17. The multi-mask equipment 100 as claimed in any one of claims 14 to 16 further comprising at least one adjusting mechanism A3 for adjusting the position of the mask assembly M on the face of the wearer.

18. The multi-mask equipment 100 as claimed in claim 17, wherein the at least one adjusting mechanism A3 comprises a plurality of belts A3, each belt A3 of the plurality of belts A3 being secured to the structured mask assembly M at one end and configured to engage with the connectors F1 on one headband F at the other end.

19. The multi-mask equipment 100 as claimed in any one of claims 17 and 18, wherein the at least one adjusting mechanism A3 comprises a plurality of teeth A4 for engaging with connectors F1 on the at least one left-side headband F and at least one right-side headband F.

20. The multi-mask equipment 100 as claimed in any one of previous claims, wherein the soft filter material D is mounted on the inner grill C1 that is covered with a gasket C2 and secured by an outer gill C3.
